(19) United States
(12) Patent Application Publication
(10) Pub. No.: US 2025/0135078 A1
(43) Pub. Date: May 1, 2025

(54) OPERATION OF A DIALYSIS SYSTEM

(71) Applicant: QUANTA DIALYSIS TECHNOLOGIES LIMITED, Warwickshire (GB)

(72) Inventors: Clive BUCKBERRY, Warwickshire (GB); Mark WALLACE, Warwickshire (GB); Henrik HALL, Warwickshire (GB)

(21) Appl. No.: 19/010,998

(22) Filed: Jan. 6, 2025

Related U.S. Application Data
(63) Continuation of application No. PCT/GB2023/051790, filed on Jul. 6, 2023.

(30) Foreign Application Priority Data
Jul. 6, 2022 (GB) 2209928.7

(51) Int. Cl. A61M 1/16

(52) U.S. Cl. CPC A61M 1/1603

(57) ABSTRACT

A system for pumping dialysis fluid comprising a dialyser having a semi-permeable membrane and at least one pump in fluid communication with the dialyser and operable to deliver a first volume of dialysis fluid from a fluid source to the dialyser in an inlet cycle. The at least one pump is operable to remove a second volume of dialysis fluid from the dialyser and deliver the second volume to a drain in an outlet cycle. The system has a controller in electrical communication with the at least one pump, the controller configured to operate the at least one pump in the inlet cycle, and operable to operate the at least one pump in the outlet cycle.

### CROSS-REFERENCE TO RELATED APPLICATIONS

This application is a continuation of International Patent Application PCT/GB2023/051790, filed Jul. 6, 2023, which claims foreign priority to United Kingdom Patent Application GB 2209928.7, filed Jul. 6, 2022, with the entirety of each of these applications hereby incorporated by reference herein.

### TECHNICAL FIELD

The present disclosure relates to operation of a dialysis system. Particularly, but not exclusively, the disclosure relates to improving dialyser performance and the efficient use of dialysis fluid. Aspects of the disclosure relate to a dialysis pumping arrangement, to a further dialysis pumping arrangement, and to a method of operating a dialysis pumping arrangement.

### BACKGROUND

Patients suffering from reduced kidney function rely on external blood treatments to remove harmful waste substances that build up in their blood over time. One of the most common methods of treatment is haemodialysis.

Haemodialysis typically involves two networks of fluid passageways running adjacent to one another in a counter-current flow arrangement. This arrangement of fluid pathways is provided in a device known as a dialyser. Blood is passed through a set of hollow fibres and dialysis fluid is passed through the spaces in between the hollow fibres. The pH and osmotic potential of the dialysis fluid is adapted such that waste compounds built up in the blood diffuse from the blood into the dialysis fluid through the walls of the hollow fibres that act as a semi-permeable membrane.

Haemodialysis provides a method of gradually removing waste materials with a molecular weight from 50 to 60000 Daltons from the blood by diffusion and convection. However, there are some challenges associated with haemodialysis.

One challenge of haemodialysis is that medium molecular weight molecules (mMW) dissolved in the blood (for example β-2 microglobulin), which are typically between 1000 and 15000 Daltons, are difficult to remove completely from the blood using haemodialysis. It can take a long time to reduce the levels of these substances in the blood to acceptable levels, which may not be convenient for a patient. One other challenge associated with haemodialysis is that molecules and proteins can become deposited and attached to the blood side of the semi-permeable membrane, causing a film to build up on the membrane over time. This is known as membrane fouling and is detrimental to the operation of the dialyser and the dialysis system as a whole.

Dialysers employing a flow arrangement may also be susceptible to concentration polarisation effects in the regions closest to the membrane. This is because, at the outer walls of hollow fibres, the laminar flow may become relatively stagnant, creating a boundary layer. Turbulent flow through this region disrupts the boundary layer.

Another phenomenon of haemodialysis utilising a dialyser as above is that the dialyser may suffer from tunnelling, where dialysis fluid tends to follow the flow path of least resistance, meaning that the entire cross-sectional area of the dialyser is not fully utilised, reducing the efficiency of the dialyser.

An alternative approach to remove waste molecules from the blood is to use a form of forced convective operation, such as haemodiafiltration.

Typically, haemodiafiltration involves administering sterile dialysis fluid to the blood either by employing a large hydrostatic potential to force sterile dialysis fluid across a semi-permeable membrane into the blood or by directly adding it to the blood; and then pulling the sterile dialysis fluid, complete with dissolved waste products, back across the semi-permeable membrane for subsequent disposal. This type of blood treatment is not limited by diffusion, as sterile dialysis fluid is allowed to mix directly with the blood and returned to the dialysis fluid by a process termed “solute drag”. Thus, the movement of waste compounds is by convection, with dialysis fluid moving across the dialyser membrane transversely. However, membrane fouling is an issue for hemodiafiltration too.

One haemodiafiltration method of directly adding dialysis fluid to the blood is controlled by infusing the blood side of the dialyser with sterile solution at a constant flow rate. However, increased patient monitoring may be necessary, and this type of treatment may not suit all patients.

There is therefore a need for improvements in improving dialyser performance and the efficient use of dialysis fluid.

### SUMMARY

Aspects and embodiments of the invention provide a system for pumping dialysis fluid, a further system for pumping dialysis fluid, a method of pumping dialysis fluid, a controller for controlling pumping of dialysis fluid and a computer program product encoded on one or more non-transitory computer storage media as claimed in the appended claims.

According to a first aspect of the invention, there is provided a system for pumping dialysis fluid, the system comprising: a dialyser having a semi-permeable membrane; at least one pump in fluid communication with the dialyser, the at least one pump operable to deliver a first volume of dialysis fluid from a fluid source to the dialyser in an inlet cycle, the at least one pump operable to remove a second volume of dialysis fluid from the dialyser and deliver the second volume of dialysis fluid to a drain in an outlet cycle; and a controller in electrical communication with the at least one pump, the controller configured to: operate the at least one pump in the inlet cycle and in the outlet cycle, operate the at least one pump to circulate dialysis fluid from the dialyser to the at least one pump and back to the dialyser in a circulation cycle isolated from the fluid source and the drain.

Optionally, the controller may be configured to operate the at least one pump with each instance of the inlet cycle corresponding to a respective instance of an outlet cycle. The controller may be configured to operate the at least one pump in the circulation cycle between completion of the inlet cycle and the outlet cycle. The operation of the at least one pump in the circulation cycle may circulate the dialysis fluid through the dialyser in a single flow direction throughout the circulation cycle.

By circulating the dialysis fluid isolated from the dialysis fluid source and the drain between the inlet and outlet pump cycles, flow through the dialyser is increased without drawing more dialysis fluid from the dialysis source. This allows more diffusion to occur across the semipermeable membrane for a given volume of fluid extracted from the dialysis fluid source. This ultimately allows less fluid to be used to remove the same amount of waste products from the patient's blood. The efficiency of the dialysis is improved.

Optionally the system may further comprise a blood pump, wherein the dialyser semi-permeable membrane divides the dialyser into a blood side and a dialysis fluid side, wherein the blood pump is in fluid communication with the blood side of the dialyser as part of a blood circuit, and the controller is configured to actuate the blood pump such that fluid flows through the blood side in a direction opposite to a flow direction in which dialysis fluid circulates through the dialysis fluid side of the dialyser throughout the circulation cycle of the at least one pump.

Since the assembly is configured such that the circulation occurs in a single flow direction throughout a given circulation cycle, a counter current flow of dialysis fluid and a patient's blood in the dialyser can be maintained throughout the circulation cycle. A counter current flow maximises the concentration gradient of solutes between the blood and the dialysis fluid and thus maximises the rate of diffusion across the semi permeable membrane.

Optionally the controller may be configured to operate the at least one pump in one or more circulation cycles between the inlet cycle and the outlet cycle.

Optionally the controller may be configured to operate the at least one pump in the circulation cycle a number of times, n, in between completion of the inlet cycle and the outlet cycle, wherein n is greater than or equal to 2.

An increase in circulation cycles makes overall flow through the fluid pathways of the system more homogeneous. Within the dialyser itself, the increased flow caused by the circulation cycles disrupt the boundary layers of the semi-permeable membrane of the dialyser and acts to prevent polarisation layers.

Optionally the controller may be configured to operate the at least one pump in the circulation cycle a predetermined number of times in between completion of the inlet cycle and the outlet cycle, wherein the predetermined number may vary according to at least one of treatment type, treatment length, and/or dialysis fluid composition.

Optionally, the controller may be further configured to reverse the single flow direction in which dialysis fluid circulates through the dialyser in a further circulation cycle. The controller may be configured to operate the at least one pump in the further circulation cycle a number of times, n, in between completion of the inlet cycle and the outlet cycle, wherein n is greater than or equal to 2.

Optionally, the at least one pump may be defined in part by a flexible membrane, the flexible membrane being independently operable between an open position and a closed position for each of the first pump and second pump.

Optionally, the system may further comprise at least one sensor in electrical communication with the controller wherein the at least one sensor is arranged to measure a signal indicative of dialysis fluid composition, the controller including instructions for receiving the signal indicative of dialysis fluid content, and instructions for carrying out operation of the at least one pump based on the dialysis fluid composition.

Optionally the system may be defined in part by one of a peristaltic pump, a double-sided piston pump, a gear pump, a vane pump or an impeller pump.

Optionally the at least one pump may be operable to move a fluid volume at a volumetric flow rate of 50 to 1000 ml/minute through the at least one pump.

According to a second aspect of the invention, there is provided a system for pumping dialysis fluid, the system comprising: a dialyser having a semi-permeable membrane; at least one pump in fluid communication with the dialyser; a fluid source in fluid communication with the at least one pump; and a drain in fluid communication with the at least one pump; the at least one pump operable to deliver a first volume of dialysis fluid from the fluid source to the dialyser in an inlet cycle, the at least one pump operable to remove a second volume of dialysis fluid from the dialyser and deliver the second volume of dialysis fluid to the drain in an outlet cycle; and a controller in electrical communication with the at least one pump, the controller configured to: operate the at least one pump in the inlet cycle and in the outlet cycle, operate the at least one pump to circulate dialysis fluid from the dialyser to the at least one pump and back to the dialyser in a circulation cycle isolated from the fluid source and the drain.

According to a third aspect of the invention, there is provided a system for pumping dialysis fluid, the system comprising: a dialyser having a semi-permeable membrane; a first pump in fluid communication between a fluid source and the dialyser; a second pump in fluid communication between the dialyser and a drain; and a controller including a processing unit and one or more non-transitory computer storage media in electrical communication with one another, the processing unit in electrical communication with the first pump and the second pump, the one or more non-transitory computer storage media having stored thereon computer-readable instructions that, when executed by the processing unit, cause the processing unit to carry out steps including: operating the first pump in an inlet cycle moving dialysis fluid from the fluid source to the dialyser, and operating the second pump in an outlet cycle moving dialysis fluid from the dialyser to the drain, and the controller coordinating operation of the first pump and the second pump in a circulation cycle fluidically isolated from the fluid source and the drain, the circulation cycle circulating dialysis fluid from the dialyser to a first one of the first pump and the second pump and back to the dialyser, and circulating dialysis fluid to the dialyser from the other of the first pump and the second pump and back to the other of the first pump and second pump.

Optionally the computer-readable instructions for causing the processing unit to operate the second pump in the outlet cycle may include computer-readable instructions for causing the processing unit to operate the second pump in the outlet cycle for each operation of the first pump in the inlet cycle.

Optionally the computer-readable instructions for causing the processing unit to coordinate operation of the first pump and the second pump in the circulation cycle may include computer-readable instructions for causing the processing unit to coordinate operation of the first pump and the second pump in the circulation cycle between completion of the inlet cycle and the outlet cycle.

Optionally the computer-readable instructions for causing the processing unit to coordinate operation of the first pump and the second pump in the circulation cycle may include computer-readable instructions for causing the processing unit to coordinate operation of the first pump and the second pump such that dialysis fluid moves in a single flow direction through the dialyser throughout the circulation cycle.

Optionally the computer-readable instructions for causing the processing unit to coordinate operation of the first pump and the second pump in the circulation cycle may include computer-readable instructions for causing the processing unit to synchronise operation of the first pump and the second pump in the circulation cycle so that when one of the first pump or the second pump delivers dialysis fluid to the dialyser, the other one of the first pump or the second pump removes dialysis fluid from the dialyser.

Optionally the system may further comprise a blood pump, wherein the dialyser semi-permeable membrane divides the dialyser into a blood side and a dialysis fluid side, wherein the blood pump is in fluid communication with the blood side of the dialyser as part of a blood circuit, and the controller is configured to actuate the blood pump such that fluid flows through the blood side in a direction opposite to a flow direction in which dialysis fluid circulates through the dialysis fluid side of the dialyser throughout the circulation cycle of the first and second pump.

Optionally the controller may be configured to operate the first pump and the second pump in one or more circulation cycles between each inlet cycle and each outlet cycle.

Optionally the controller may be configured to operate the first pump and the second pump in the circulation cycle a number of times, n, in between completion of the inlet cycle and the outlet cycle, wherein n is greater than or equal to 2.

By operating the each of the first pump and the second pump in their own circulation cycles, flow through the dialyser is greatly increased. This allows for flow to be maintained through the dialyser between completion of the inlet cycle and outlet cycle. By operating each of the first pump and the second pump in their own cycles, it can be arranged that there is no moment in which fluid in the dialyser is static. This greatly improves the efficiency of dialysis.

Optionally the controller may be configured to operate the first pump and the second pump in the circulation cycle a predetermined number of times in between completion of the inlet cycle and the outlet cycle, wherein the predetermined number may vary as a function of at least one of treatment type, treatment length, and/or dialysis fluid composition.

Optionally the first pump and the second pump may be both operable to deliver a first volume of dialysis fluid from a dialysis fluid source to the dialyser in an inlet cycle, and both operable to remove a second volume of dialysis fluid from the dialyser and deliver the second volume of dialysis fluid away from the dialyser in an outlet cycle.

Optionally the controller may be further configured to reverse the single flow direction in which dialysis fluid circulates through the dialyser in a further circulation cycle. Optionally the controller may be configured to operate the first pump and the second pump in the further circulation cycle a number of times, n, in between completion of the inlet cycle and the outlet cycle, wherein n is greater than or equal to 2.

Optionally each of the first pump and the second pump are defined in part by a flexible membrane, the flexible membrane being independently operable between an open position and a closed position for each of the first pump and second pump.

Optionally the first pump further comprises a first inlet valve, and a first outlet valve, wherein the first inlet valve is arranged between the first pump and a dialysis fluid inlet of the dialyser, and the first outlet valve is arranged between a dialysis fluid outlet of the dialyser and the first pump, wherein the first inlet valve and the first outlet valve are in electrical communication with the controller and the controller includes instructions for controlling the first inlet valve and the first outlet valve independently.

Optionally the second pump further comprises a second inlet valve, and a second outlet valve, wherein the second inlet valve is arranged between the second pump and a dialysis fluid inlet of the dialyser and the second outlet valve is arranged between a dialysis fluid outlet of the dialyser and the second pump, wherein the second inlet valve and the second outlet valve are in electrical communication with the controller and the controller includes instructions for controlling the second inlet valve and the second outlet valve independently.

Optionally a first fluid source valve may be arranged between the fluid source and the first pump, wherein first fluid source valve is in electrical communication with the controller and the controller includes instructions for controlling the first fluid source valve independently.

Optionally a first drain valve may be arranged between the second pump and the drain, wherein first drain valve is in electrical communication with the controller and the controller includes instructions for controlling the first drain valve independently.

Optionally, the step of operating at least one pump in an inlet cycle to deliver a first volume of a dialysis fluid from a dialysis fluid source to a dialyser having a semi-permeable membrane may comprise operating a first pump; the step of operating the at least one pump in an outlet cycle to remove a second volume of the dialysis fluid from the dialyser to a drain may comprise operating a second pump; and the steps of operating the at least one pump to circulate the dialysis fluid from the dialyser to the at least one pump and back to the dialyser in a circulation cycle isolated from the dialysis fluid source and the drain may comprise operating the first pump and the second pump to circulate the dialysis fluid from the dialyser to a first one of the first pump and the second pump and back to the dialyser, and to circulate dialysis fluid to the dialyser from the other of the first pump and the second pump and back to the other of the first pump and the second pump in a circulation cycle isolated from the dialysis fluid source and the drain.

Optionally, the method may further comprise the step of: operating a blood analogue pump, wherein the dialyser semi-permeable membrane divides the dialyser into a blood analogue side and a dialysis fluid side, wherein the blood analogue pump is in fluid communication with the blood side of the dialyser as part of a blood analogue circuit; wherein operating the blood analogue pump causes a blood analogue fluid to flow through the blood analogue side in a flow direction opposite to the single flow direction in which the cleaning fluid circulates throughout the circulation cycle.

The blood analogue fluid is a fluid designed to behave in a similar fashion to blood to enable the device to be operated and tested. Blood plasma may be used as the analogue fluid. The blood analogue fluid preferably includes a marker, such as a dye or a marker molecule which may be sensed using appropriate sensors.

Optionally, the circulation may Optionally, the operations of the first pump and second pump may be synchronised in the circulation cycle so that when one pump delivers dialysis fluid to the dialyser, the other pump removes dialysis fluid from the dialyser.

Optionally, the method may further comprise repeating the circulation cycle a number of times, n, in between completion of the inlet cycle and the outlet cycle, wherein n is greater than or equal to 2. Optionally, the circulation may occur in the same flow direction for each circulation cycle occurring between completion of a given inlet cycle and outlet cycle.

Optionally, the method may further comprise the step of reversing the single flow direction in which cleaning fluid circulates in the further circulation cycle.

Optionally, the method may further comprise the step of alternating the pump which performs the inlet cycle and alternating the pump that performs the outlet cycle after a fixed number of inlet and outlet pump cycles, N, optionally where N=20.

According to a fifth aspect of the present invention, there is provided a method of pumping dialysis fluid, the method comprising the steps of: operating a first pump to deliver a first volume of a dialysis fluid to a dialyser and operating a second pump to remove a second volume of dialysis fluid from the dialyser; operating the second pump to deliver the second volume of dialysis fluid back to the dialyser in a first circulation cycle and operating the first pump to remove a third volume of the dialysis fluid from the dialyser; and operating the first pump to deliver the third volume of dialysis fluid back to the dialyser in a second circulation cycle and operating the second pump to remove a fourth volume of the dialysis fluid from the dialyser, wherein the first and second circulation cycles occur in a single flow direction.

Optionally, the method may comprise the further step of operating the first pump to draw a further volume of dialysis fluid from a dialysis fluid source and operating the second pump to deliver the fourth volume of dialysis fluid to drain.

Optionally, the method may further comprise the step of: operating a blood analogue pump, wherein the dialyser semi-permeable membrane divides the dialyser into a blood analogue side and a dialysis fluid side, wherein the blood analogue pump is in fluid communication with the blood side of the dialyser as part of a blood analogue circuit; wherein operating the blood analogue pump causes a blood analogue fluid to flow through the blood analogue side in a flow direction opposite to the single flow direction in which the cleaning fluid circulates throughout the circulation cycle.

### BRIEF DESCRIPTION OF THE DRAWINGS

One or more embodiments will now be described, by way of example only, with reference to the accompanying drawings, in which:

FIG. 1 is a schematic representation of a system for pumping dialysis fluid;

FIG. 2 is a partial enlarged view of a dialyser the system of FIG. 1;

FIGS. 3 to 8 are idealised graphical representations of dialysis fluid flow direction at the dialyser as a function of time;

FIG. 9 is the schematic representation of the system of FIG. 1 highlighting a first circulation path;

FIG. 10 is the schematic representation of the system of FIG. 1 highlighting a second circulation path;

FIG. 11 is a schematic representation of a system for pumping dialysis fluid;

FIG. 12 is a schematic representation of a system for pumping dialysis fluid;

FIG. 13 is a schematic representation of a membrane pump; and

FIG. 14 is a flowchart of a method of pumping dialysis fluid in accordance with an example implementation.

### DETAILED DESCRIPTION

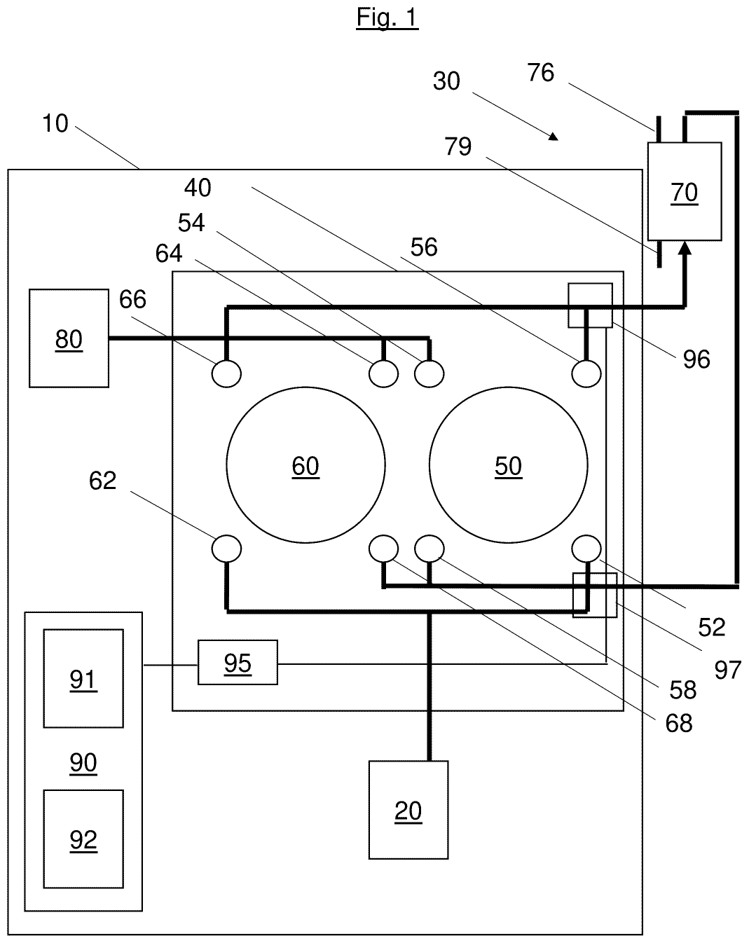

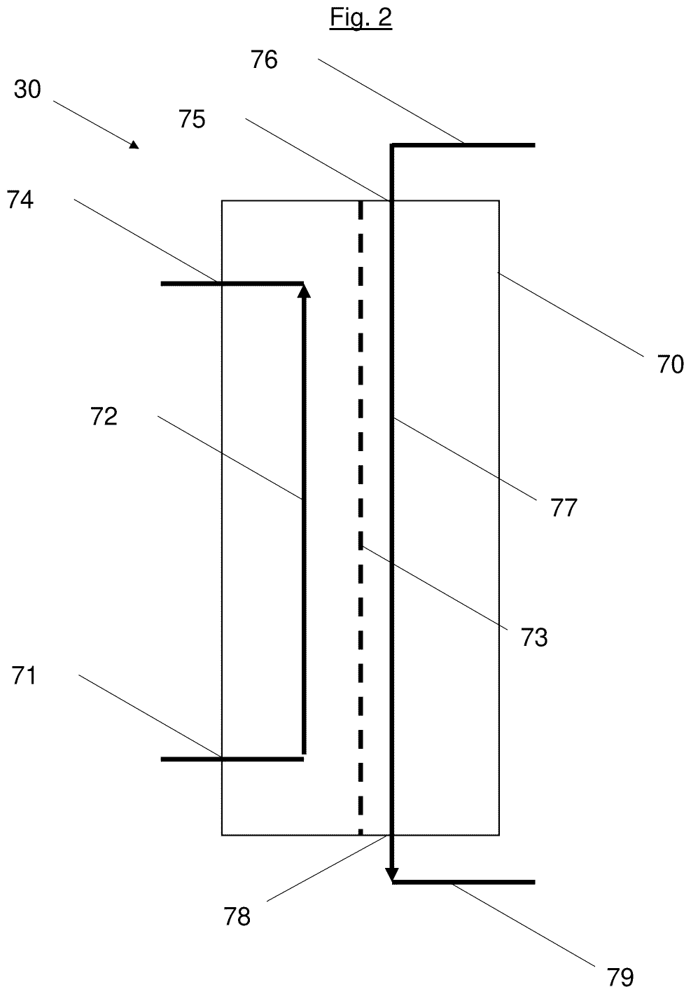

Referring to FIGS. 1 and 2, a system for pumping dialysis fluid, (hereinafter system 10), includes at least in part, a dialysis fluid source 20, a dialysis fluid cassette 40, a dialyser 70, a drain 80 and a controller 90. The controller 90 has a processing unit 91 and a memory 92.

The dialysis fluid cassette 40 defines the fluid pathways, pumps and valves required to move dialysis fluid from the fluid source 20 to the dialyser 70 and further to the drain 80. The dialysis fluid cassette 40 is receivable within a dialysis machine such that the pumps and valves may be actuated appropriately, with the controller 90 being in electrical communication with pumps and valves of system 10.

The dialysis fluid cassette 40 defines a first flow balance pump 50 and a second flow balance pump 60. Each of the first flow balance pump 50 and the second flow balance pump 60 has a fluid source inlet valve 52, 62 (respectively), a drain valve 54, 64 (respectively), a dialyser inlet valve 56, 66 (respectively), and a dialyser outlet valve 58, 68 (respectively). The pumps and valves are membrane pumps and valves and formed between flexible membrane of the dialysis fluid cassette 40. An example of a dialysis fluid cassette is set out in WO2006120415, entitled Dialysis Machine, the entire contents of which are hereby incorporated herein by reference. Another example of a dialysis fluid cassette is set out in WO2010146344, entitled Dialysis Machine Control, the entire contents of which are hereby incorporated herein by reference.

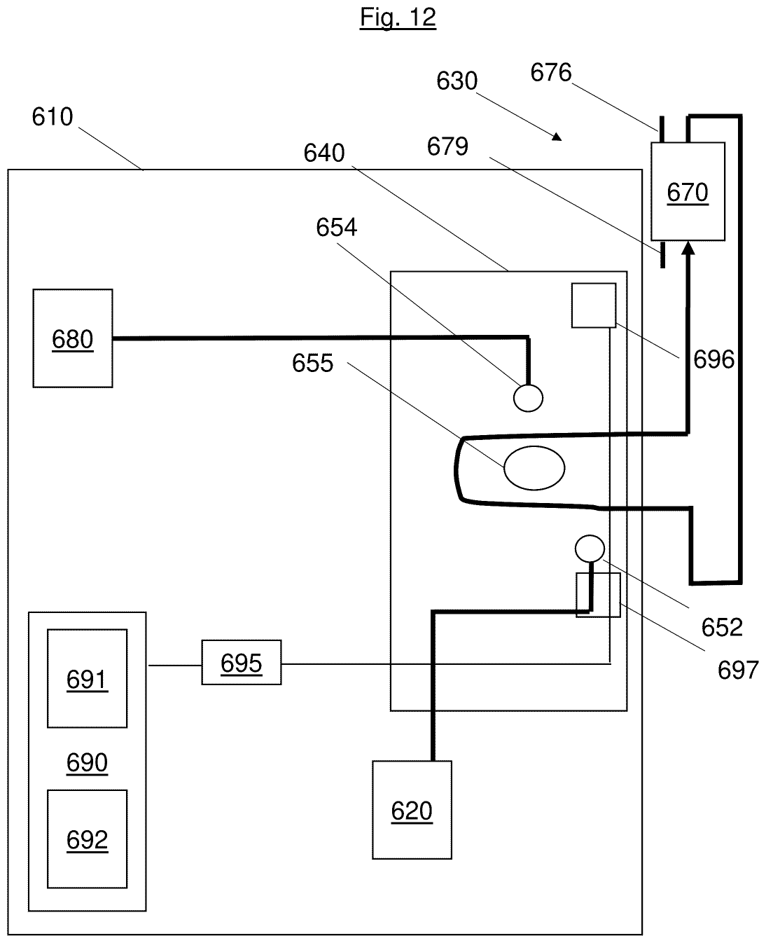

Referring to FIG. 13, an exemplary membrane pump 700 is shown. The dialysis fluid cassette 40 has a recessed surface 42 and a flexible membrane 44, with a pump chamber 46 is defined therebetween. The pump chamber 46 us in fluid communication with a common inlet and outlet 48.

The dialysis fluid cassette 40 abuts a pump driver 710 comprising a platen 720 having a recessed surface 730 therein and a fluid port 740. In use, the platen 720 is kinematically located against the dialysis fluid cassette 40 to sealingly engage with the dialysis fluid cassette 40 such that the recessed surface 730 and the flexible membrane 44 define a drive chamber 740.

The fluid port 740 is connectable with a source of positive fluid pressure 750 and a negative source of fluid pressure 760 via a modulated valve 770, controlled by the controller 90 to allow fluid to flow into or out of the drive chamber 740. The modulated valve 770 is a proportional valve having a variable sized orifice therein, the valve being controllable to change the size of the orifice, thereby controlling the flow of fluid therethrough.

The sources of positive and negative fluid pressure 750, 760 include a pressure pump and a vacuum pump respectively. When the modulated valve 770 is operated to allow fluid to flow into the drive chamber 610 from the source of positive fluid pressure 750, the flexible membrane 44 moves towards the recessed surface 42 and any dialysis fluid that is in the pump chamber 46 is pumped out of the common inlet and outlet 48. When the modulated valve 770 is operated to allow fluid to flow out of the drive chamber 610 to the source of negative fluid pressure 760, the flexible diaphragm 44 is moved away from the recessed surface 42 towards surface 730 and dialysis fluid is drawn into the pump chamber 46 from the common inlet and outlet 48. As such the flexible membrane is independently operable between an open position 44A and a closed position 44B.

Referring to FIG. 2A blood circuit 30 comprises a venous line 79 connected to the dialyser 70 and an arterial line 76 connected to the dialyser 70. Blood moves through the blood circuit 30 under action of a blood pump. The controller 90 is configured to actuate the blood pump. The view of the dialyser 70 is schematic in nature and does not show the individual hollow fibres and their spatial arrangement. An example of haemodialysis is set out in WO2015/022537, entitled Dual Haemodialysis and Haemodiafiltration Blood Treatment Device, the entire contents of which are hereby incorporated herein by reference.

Methods of operating the system are disclosed and in such a case, the blood from a patient may be replaced by a source of blood analogue fluid. The blood analogue fluid is a fluid which behaves in a similar fashion to blood. The blood analogue fluid may include markers such as a dye to allow an observer to confirm firstly that components of the blood analogue fluid interact appropriately with the blood fluid circuit and dialyser 70, and secondly that semi-permeable membrane of the dialyser 70 may be cleaned, as described in more detail below. The blood analogue fluid may be Dextran with marker molecules, such as fluorescein markers, which can be sensed using an appropriate sensor, such as a fluorometer. This facilitates testing the system to confirm that fluid is passed from the blood side of the semi-permeable membrane to the dialysate side of the semi-permeable membrane and that relevant molecules were removed from the blood analogue fluid when passed back across the semi-permeable membrane from the dialysate side to the blood side of the system. An example of operating a dialysis system is set out in WO2018/142153, entitled Phased Convective Operation, the entire contents of which are hereby incorporated herein by reference.

Dialysis fluid enters the dialyser 70 at a dialysis fluid inlet 71, passes through the dialyser 70 in a first direction (indicated by arrow 72) on one side of a semi-permeable membrane 73 and exits through a dialysis fluid outlet 74. Blood or blood analogue fluid enters the dialyser 70 through an arterial inlet 75, via the arterial line 76, passes through the dialyser 70 in a second direction (indicated by arrow 77 and opposite the first direction) on another side of the semi-permeable membrane 73 and exits the dialyser 70 through a venous outlet 78 into the venous line 79. Hence the relative flow of dialysis fluid and blood fluid is in opposite directions along the semi-permeable membrane 73.

To achieve an overall mean dialysis fluid flow of 50 ml/min at the dialyser 70, the number of pump pulses occurring in a given time period must be reduced. FIG. 4 shows such an exemplary lower flow rate pump operation whereby there are two synchronised pump pulses of the first flow balance pump 50 and the second flow balance pump 60 in the manner of the first step, collectively numbered 201 and 211. In this sense 101 and 201 are equivalent, and 111 and 211 are equivalent.

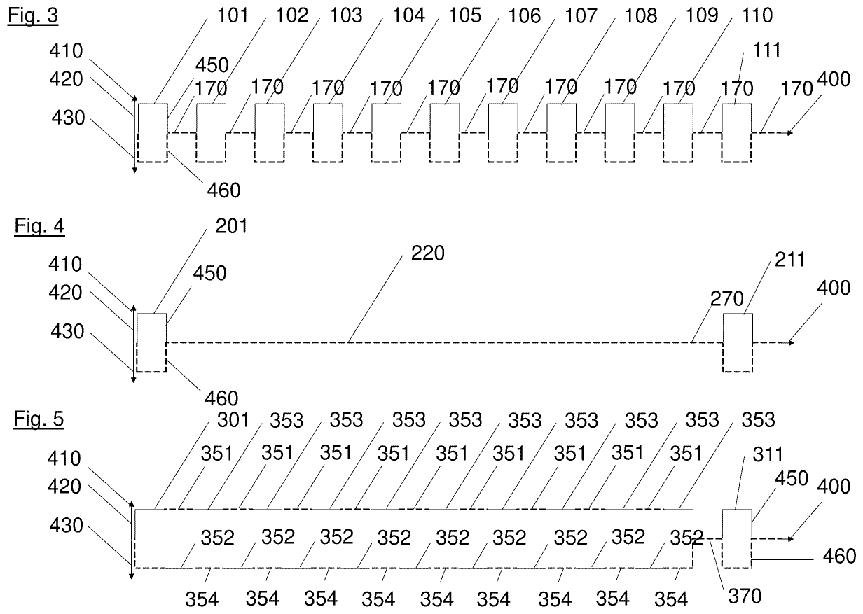

Referring back to FIG. 3, for simplicity, the first ten pump pulses 101 to 110 occur over a period of time, t. This can be compared directly with FIG. 4, whereby there is only a single pump pulse 201 for the same period of time, t. Equally, there is only a “single” flat portion 270.

As such this results in an overall mean dialysis fluid flow of 50 ml/min at the dialyser 70. The overall mean dialysis fluid flow from the source and the overall mean dialysis fluid flow to the drain is also 50 ml/min. This is evident, since for every ten pump pulses of FIG. 3, there is only a single pump pulse in FIG. 4. As such the duty cycle represented in FIG. 4 is 5%.

Comparing FIGS. 3 and 4 demonstrates how the same pump pulses and same pump pressures may be used for different flow rates, through altering the duty cycle.

As can be seen in the 5% duty cycle pump operation, there is a significant gap 220 between the first pump pulse 201, the single flat portion 270 and the second pump pulse 211. This gap 220 is known as the dwell time, where both pumps 50, 60 are inactive. In the gap 220 the dashed line 460 is overlying and obscuring the solid line 450. FIG. 5 will be described in more detail below.

### Circulating Flow

As described above, the first flow balance pump 50 is operable to deliver a first volume of dialysis fluid from the dialysis source 20 to the dialyser 70 in an inlet cycle (pumping step 1 and step 2). The second flow balance pump 60 is operable to remove a second volume of dialysis fluid from the dialyser 70 and deliver the second volume of dialysis fluid to the drain 80 in an outlet cycle (pumping step 1 and step 2). The controller 90 is configured to operate the first flow balance pump 50 in the inlet cycle, and operable to operate the second flow balance pump 60 in the outlet cycle. For example, for each inlet cycle, there may be a corresponding outlet cycle.

The controller 90 is further configured to operate the first flow balance pump 50 and the second flow balance pump 60 to circulate dialysis fluid from the dialyser 70 to a first one of the first flow balance pump 50 and the second flow balance pump 60 and back to the dialyser 70 and to circulate dialysis fluid to the dialyser from the other of the first flow balance pump 50 and the second blow balance pump 60 and back to the other of the first flow balance pump 50 and the second flow balance pump 60 in a circulation cycle isolated from the fluid source 20 and the drain 80. The circulation cycle may occur between completion of the inlet cycle and the outlet cycle. Further, or instead, circulation may occur in a single flow direction through the dialyser 70 throughout a given circulation cycle. This is exemplified in circulation steps 1 and 2 described below.

With the first flow balance pump 50 full of fresh dialysis fluid, and with the second flow balance pump 60 empty, the following pumping operations are performed.

In circulation step 1, dialysis fluid is pumped from the first flow balance pump 50 to the dialyser 70 via the inlet valve 56, by actuation of the first flow balance pump 50. Spent dialysis fluid is drawn from the dialyser 70 into the second flow balance pump 60 via the outlet valve 68, by actuation of the second flow balance pump 60.

At the end of the circulation step 1, the first flow balance pump 50 is empty, and the second flow balance pump 60 is full of spent dialysis fluid.

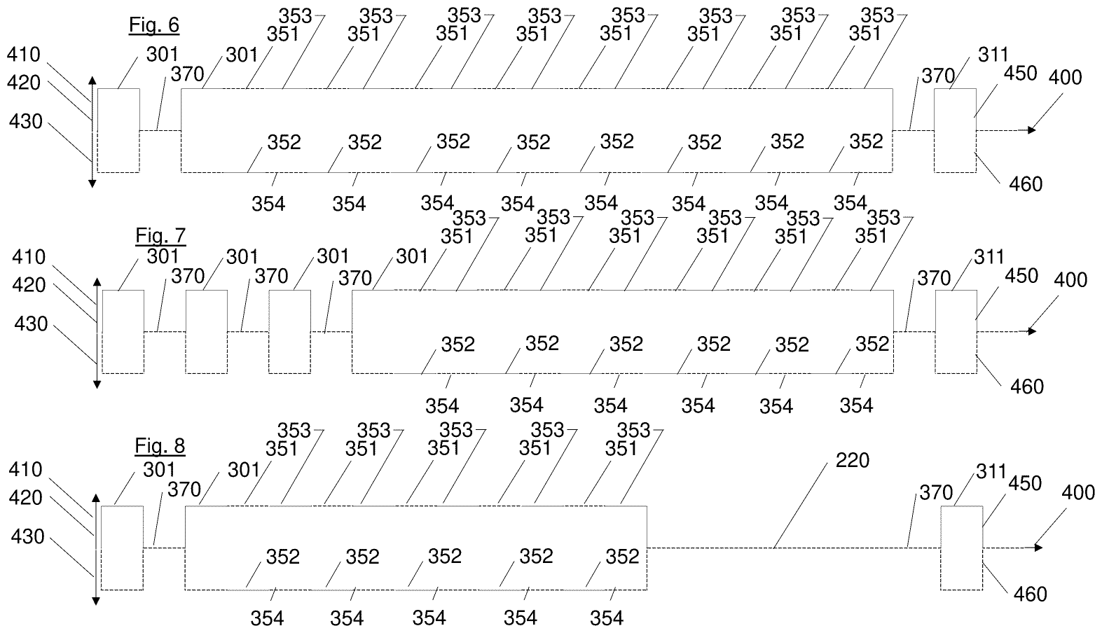

In circulation step 1, the spent dialysis fluid is pumped from the second flow balance pump 60 to the dialyser 70, via the inlet valve 66, by actuation of the second flow balance pump 60, whilst spent dialysis fluid is drawn from the dialyser 70 into the first flow balance pump 50, via dialyser outlet valve 58, by actuation of the first flow balance pump 50. This is shown in FIG. 9.

Thus, the spent dialysis fluid is pumped back into the dialyser 70, but in the same flow direction as fresh dialysis fluid. This maintains the counter-current flow of dialysis fluid and blood.

At the end of circulation step 1, the first flow balance pump 50 is full of spent dialysis fluid, and the second flow balance pump 60 is empty.

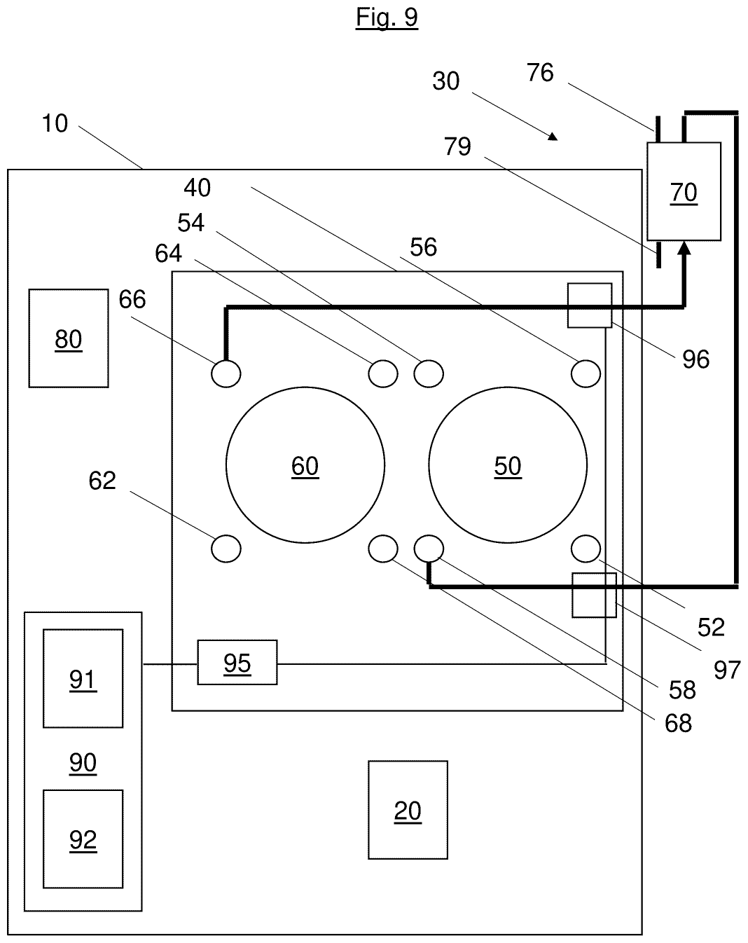

In circulation step 2, the spent dialysis fluid is pumped from first flow balance pump 50 to the dialyser 70, via the inlet valve 56, by actuation of the first flow balance pump 50. Spent dialysis fluid is drawn from the dialyser 70 into the second flow balance pump 60, via dialyser outlet valve 68, by actuation of the second flow balance pump 60. This is shown in FIG. 10.

Thus, again the spent dialysis fluid is pumped back into the dialyser 70, but in the same flow direction as fresh dialysis fluid. This maintains the counter-current flow of dialysis fluid and blood.

In the circulation steps 1 and 2, the first flow balance pump 50, the second flow balance pump 60 and the dialyser 70 define a closed fluid circuit, fluidically isolated from the fluid source 20 and the drain 80.

At the end of circulation step 2, first flow balance pump 50 is empty, and the second flow balance pump 60 is full of spent dialysis fluid, in a similar condition to the beginning of circulation step 1. Because the flow balance pump conditions are the same as for the beginning of circulation step 1, circulation steps 1 and 2 may be repeated.

In circulation step 2, fresh dialysis fluid is drawn from fluid source 20 into the first flow balance pump 50, via the fluid source inlet valve 52, by actuation of the first flow balance pump 50. The spent dialysis fluid is pumped from the second flow balance pump 60 to the drain 80, via the drain valve 64, by actuation of the second flow balance pump 60.

Therefore, in use, the above operation switches between an open dialysis fluid flow path and a closed fluid circuit dialysis fluid flow path. Continuous flow of dialysis fluid through the dialyser 70 is maintained in the same direction of flow. This is achieved by creating a circulating flow path fluidically isolated from the drain 80 and the fluid source 20.

Circulation steps 1 and 2 may collectively be termed a circulation cycle. For each inlet cycle and each outlet cycle there is at least one circulation cycle. The circulation cycle may be repeated a number of times, n, between the start and completion of each inlet cycle and each outlet cycle, n being a number that is greater than or equal to 2. This is exemplified by the repetition of circulation steps 1 and 2 as per Table 1 below. The number of times, n, may be stored in the memory 92 of controller 90. Alternatively, the number of times, n, may be calculated by the processor 91 of controller 90 dependent upon the operating conditions of the system 10. The operating conditions may include treatment type, treatment length, and/or dialysis fluid composition.

TABLE 1
Repetition of circulation pump cycle

Pump Pulse

| | Pump A | Pump B | Step |
|---|---|---|---|
| 301 | | | Pumping Step 1 |
| | 351 | 352 | Circulation Step 1 |
| | 353 | 354 | Circulation Step 2 |
| | 351 | 352 | Circulation Step 1 |
| | 353 | 354 | Circulation Step 2 |
| | 351 | 352 | Circulation Step 1 |
| | 353 | 354 | Circulation Step 2 |
| | 351 | 352 | Circulation Step 1 |
| | 353 | 354 | Circulation Step 2 |
| | 351 | 352 | Circulation Step 1 |
| | 353 | 354 | Circulation Step 2 |
| | 351 | 352 | Circulation Step 1 |
| | 353 | 354 | Circulation Step 2 |
| | 351 | 352 | Circulation Step 1 |
| | 353 | 354 | Circulation Step 2 |
| | 351 | 352 | Circulation Step 1 |
| | 353 | 354 | Circulation Step 2 |
| | 351 | 352 | Circulation Step 1 |
| | 353 | 354 | Circulation Step 2 |

| | |
|---|---|
| 370 | Pumping Step 2 |

The repetition of the circulation steps 1 and 2 may be a predetermined number of times in between completion of the inlet cycle and outlet cycle. The predetermined number may vary as a function of at least one of treatment type, treatment length, or under closed loop control with feedback determined by dialysis fluid composition, as sensed by the sensors 95 on the dialysis fluid cassette 40.

The circulation cycle may be repeated a number of times, n, in between the completion of each inlet cycle and each outlet cycle, wherein n corresponds to substantially continuous flow through the dialyser.

Optionally, pump swapping may also, or instead, be used. This includes alternating the roles of the first flow balance pump 50 and second flow balance pump 60 in a similar manner as explained above. By alternating the roles, the direction of circulation of dialysis fluid may also be changed, such that the circulation cycle may occur in a counter flow with respect to the flow of blood, or in the same direction as the flow of blood.

In the graphical representation of dialysis fluid flow at the dialyser 70 of FIG. 5, the effect of circulation steps 1 and 2 are shown. Comparing FIGS. 3, 4 and 5 demonstrates how circulating flow may be used when the duty cycle is reduced. As was described above, there is a gap 220 (a dwell time) during which there is no flow, in the dialyser 70, between the first pump pulse 201 and the second pump pulse 211. In the dialysis fluid flow direction of FIG. 5, circulating flow is used. That is, there are two synchronised pump pulses 301, 311 of the first flow balance pump 50 and the second flow balance pump 60 with a 5% duty cycle. Subsequent to the pumping step 1 corresponding to the first pump pulse 301, circulation steps 1 and 2 are repeated nine times to make use of all of the available dwell time ahead of the stationary portion 370. This is listed as synchronised pump pulses 351 and 353 in Table 2. It is possible to use some or all of the dwell time.

In circulation step 1, the spent dialysis fluid is pumped from the second flow balance pump 60 to the dialyser 70, via the dialyser inlet valve 66, by actuation of the second flow balance pump 60 (shown as pumping pulse 351) whilst spent dialysis fluid is drawn from the dialyser 70 into the first flow balance pump 50, via the dialyser outlet valve 58, by actuation of the first flow balance pump 50 (shown as pumping pulse 352). In circulation step 2, the spent dialysis fluid is pumped from the first flow balance pump 50 to the dialyser 70 (shown as pumping pulse 353). Spent dialysis fluid is drawn from the dialyser 70 into the second flow balance pump 60 (shown as pumping pulse 354).

Fresh dialysis fluid is drawn from fluid source 20 into the first flow balance pump 50 via dialysis fluid source inlet valve 52, by actuation of the first flow balance pump 50. The spent dialysis fluid is pumped from second flow balance pump 60 to the drain 80 via the drain valve 64, by actuation of the second flow balance pump 60 (step 2). Note these pumping operations are not shown on FIG. 5 as pump pulses, since FIG. 5 shows only fluid flow to and from the dialyser. The stationary flat portion of FIG. 5, 370, represents the filling of the first flow balance pump 50 with fresh dialysis fluid from the fluid source 20 and the emptying of the second flow balance pump 60 of the spent dialysis fluid to the drain 80. This is analogous to stationary flat portions 170 of FIG. 3.

The dialyser 70 may have a dialysis fluid capacity of 110 ml. The maximum dialysis fluid volume of the first flow balance pump 50 and the second flow balance pump 60 is 22 ml each. However, only one of the first flow balance pump 50 and the second flow balance pump 60 is full at any one time. The approximate volume of tubing in the dialysis fluid circuit, between the dialyser and one of the first flow balance pump 50, or the second flow balance 60 is 9 ml. Therefore, the approximate wetted volume of the dialysis fluid circuit is 150 ml, in a circulating flow condition.

In step 1 and step 2 of the pumping cycle described above, for each pump of the first flow balance pump 50 towards the dialyser 70, approximately 13 ml of fresh dialysis fluid is delivered to the dialyser 70, whereas 9 ml of fresh dialysis fluid remains in the tubing, between the first flow balance pump 50 and the dialyser 70, until the next operation of the first flow balance pump 50 in step 1 in a subsequent cycle. When using circulating flow, circulating flow steps 1 and 2, this is no longer the case. This provides a more efficient use of dialysis fluid and mitigates the need to close couple the dialyser 70, the first flow balance pump 50 and the second flow balance pump 60. Arranging a dialysis machine with a close coupled dialyser 70 may be physically difficult for a caregiver or indeed patient. Furthermore, such a close coupled relationship may restrict the choice of dialyser model, which in turn may restrict the treatment regimens under which the dialysis machine may be operated.

FIGS. 6, 7 and 8 show the use of circulation with different duty cycles. Similar reference numbers are used in these figures to represent similar features of FIGS. 3, 4 and 5.

In FIG. 6, the duty cycle is doubled, as compared to FIG. 5, from 5% to 10%. That is, there are two synchronised pump pulses of the first flow balance pump 50 and the second flow balance pump 60, numbered 301, with a 10% duty cycle. Subsequent to the pumping step 1 corresponding to the second pump pulse 301, circulation step 1 (351, 352) and circulation step 2 (353, 354) are repeated eight times to make use of the available dwell time (e.g., to make use of all of the available dwell time). The two synchronised pump pulses 301 of the first flow balance pump 50 and second flow balance pump 60, may occur sequentially, as shown in FIG. 6, or at any time during the overall time period.

In FIG. 7, the duty cycle is quadrupled, as compared to FIG. 5, from 5% to 20%. That is, there are four synchronised pump pulses of the first flow balance pump 50 and the second flow balance pump 60, numbered 301, with a 20% duty cycle. Subsequent to the pumping step 1 corresponding to the fourth pump pulse 301, circulation step 1 (351, 352) and circulation step 2 (353, 354) are repeated six times to make use of the available dwell time (e.g., to make use of all of the available dwell time). The four synchronised pump pulses 301 of first flow balance pump 50 and the second flow balance pump 60, may occur sequentially, as shown in FIG. 7, or at any time during the overall time period.

In FIG. 8, the duty cycle is the same as per FIG. 6. That is, there are two synchronised pump pulses of the first flow balance pump 50 and the second flow balance pump 60, numbered 301, with a 10% duty cycle. Subsequent to the pumping step 1 corresponding to the second pump pulse 301, circulation step 1 (351, 352) and circulation step 2 (353, 354) are repeated five times, with the remainder of the gap 220 representing dwell time, in which both the first flow balance pump 50 and the second flow balance pump 60 are inactive. In the gap 220 in FIG. 8, the dashed line 460 is overlying and obscuring the solid line 450. The two synchronised pump pulses 301 of the first flow balance pump 50 and the second flow balance pump 60, may occur sequentially, as shown in FIG. 8, or at any time during the overall time period. The dwell time may occur before, after or between the circulation steps 1 and 2.

### Flow Regimens

Exemplary flow regimens 1 to 6 are presented, each of which are with respect to the system 10 of FIG. 1. hereinbefore described.

### Example 1—Single Pass Flow (500 ml/Min)—as Shown in FIG. 3

An overall mean dialysis fluid flow of 500 ml/min through the dialyser 70.

An overall mean dialysis fluid flow from the fluid source 20 is also 500 ml/min.

An overall mean dialysis fluid flow to the drain 80 is also 500 ml/min.

The first flow balance pump 50 and the second flow balance pump 60 each operate with a duty cycle of 50%.

In a single-pass operation (steps 1 and 2 above-without any circulation steps), the flow through the dialyser 70 and on to drain 80 averages 500 ml/minute. In this example, this is achieved with the first flow balance pump 50 and the second flow balance pump 60 each operating with fill and empty rates of 1000 ml/minute, where the first flow balance pump 50 and the second flow balance pump 60 are each operated for 50% of the time, as shown in FIG. 3.

For the total dialysis circuit volume of 150 ml:

In 1 minute at 500 ml/min mean dialysate flow, the semi-permeable membrane 73 of the dialyser 70 is equivalently “washed” by dialysis fluid (1000 ml/min×50%/150 ml) 3.33 times.

### Example 2—Single Pass Flow (50 ml/Min)—as Shown in FIG. 4

An overall mean dialysis fluid flow of 50 ml/min through the dialyser 70.

An overall mean dialysis fluid flow from the fluid source 20 is also 50 ml/min.

An overall mean dialysis fluid flow to the drain 80 is also 50 ml/min.

The first flow balance pump 50 and the second flow balance pump 60 each operate with a duty cycle of 5%.

In a single-pass operation (steps 1 and 2 above—without any circulation steps) the flow through the dialyser 70 and to the drain 80 averages 50 ml/minute. This is achieved with the first flow balance pump 50 and the second flow balance 60 fill and empty rate of 1000 ml/minute, where the first flow balance pump 50 and the second flow balance pump 60 are operated for 5% of the time, as shown in FIG. 4.

For the total dialysis circuit volume of 150 ml:

In 1 minute at 500 ml/min mean dialysate flow, the semi-permeable membrane 73 of the dialyser 70 is equivalently “washed” by dialysis fluid (1000 ml/min×5%/150 ml) 0.33 times.

### Example 3—Single Pass Flow (50 Ml/Min) in Combination with Multiple Pass Circulating Flow—as Shown in FIG. 5

An overall mean dialysis fluid flow of 1000 ml/min through the dialyser 70.

An overall mean dialysis fluid flow from the fluid source 20 is 50 ml/min.

An overall mean dialysis fluid flow to the drain 80 is also 50 ml/min.

The first flow balance pump 50 and the second flow balance pump 60 each operate with a duty cycle of 100%.

As described above, and in comparing FIGS. 4 and 5, with the first flow balance pump 50 and the second flow balance pump 60 having fill and empty rates of 1000 ml/minute, the first flow balance pump 50 and the second flow balance pump 60 are operated for one tenth of the time, and the remaining nine-tenths of the time is available for circulating flow.

For the total dialysis circuit volume in a circulating flow condition of 150 ml:

In 1 minute at 1000 ml/min mean dialysate flow, the semi-permeable membrane 73 of the dialyser 70 is equivalently “washed” by dialysis fluid (1000 ml/min×100%/150 ml) 6.66 times given circulation steps 1 and 2 (repeated 9 times).

As may be appreciated from this example, the continuous circulating flow washes the semi-permeable membrane 73 of the dialyser 70 twice as much as single-pass dialysate flow (Example 1) while consuming only a small amount of dialysis fluid-namely, consuming the same amount of dialysis fluid as single-pass flow with the first flow balance pump 50 and the second flow balance pump 60 each operating at 5% duty cycle (Example 2).

Washing the dialyser more frequently facilitates achieving a higher osmotic level in the dialysis fluid relative to the blood than in single-pass haemodialysis. In fact, the more circulating flow that is used, the closer the dialysis fluid composition may approach to equilibrium with the blood. In effect, as compared to single-pass haemodialysis, a greater proportion of the osmotic potential of the dialysis fluid is utilised.

As compared to single-pass haemodialysis, this closed-loop arrangement improves clearance of middleweight toxin molecules, and slows the drive of patient sodium and electrolytes over the therapy time, which increases the likelihood of reducing intradialytic hypotension. Further, or instead, the closed-loop arrangement significantly reduces water consumption, as compared to single-pass haemodialysis. This is because water is only used to replenish fresh dialysis fluid, and not when the spent dialysis fluid is pumped around the closed circuit. This facilitate supplying dialysis fluid from bags, rather than from a plumbed water source, making haemodialysis treatment more available to patient populations in areas with little or no water infrastructure.

Given the membrane pump behaviours of pumping in fluid packets, rather than a continuous flow, when the dialysis system is operated at low flow rates there may be a large amount of pump down time when the pump is not in use. Nevertheless, the dialysis fluid within the packet is turbulent, which facilitates continual mixing. However, the additional pumping operations described above also leverage that turbulent flow to disrupt the boundary layers further, reducing the likelihood of tunnelling effects through the dialyser 70 and also to make the dialysis fluid flow more homogeneous. The pulsatile nature of continuous flow delivers turbulence and fluid hammer to the hollow fibres making up the dialyser semi-permeable membrane 73.

The repetition of circulation steps 1 and 2 may be the same number for each overall pumping cycle. It may not be necessary to utilise the entire dwell time 220 for circulation. Available dwell time is dependent upon the duty cycle of the particular flow regimen, driven by the target flow rate of the dialysis fluid. The repetition can be varied. For example, 8 repetitions of circulation steps 1 and 2 in the single-pass flow direction of the dialysis fluid, followed by 8 repetitions of modified circulation steps 1 and 2 in an opposite flow direction (which is in the same direction as the blood flow, rather than in a counter flow direction). Such a flow regimen may be especially effective at reducing the likelihood of tunnelling, disrupting the boundary layer and reducing the likelihood of building up polarisation layers. This is due to the increased disruption and perturbance of the semi-permeable membrane 73 of the dialyser 70. A slower drive of patient sodium and electrolytes over the therapy time will tend to reduce intradialytic hypotension (IDH).

Examples 4, 5 and 6 demonstrate the flexibility to increase and or decrease circulation during dwell periods, and that dwell periods themselves may be changed. Examples 4 and 5 demonstrate the variation in circulation according to the available dwell period. Example 6 demonstrates that not all of the dwell period needs to be dedicated to circulation. The flow parameters of Examples 1 to 6 are summarised in Table 2 below.

TABLE 2
Summary of Flow Regimens - the first flow balance
pump 50 and the second flow balance pump 60
fill and empty rate of 1000 ml/minute.

| | Exam- | Exam- | Exam- | Exam- | Exam- | Exam- |
|---|---|---|---|---|---|---|
| | ple 1 | ple 2 | ple 3 | ple 4 | ple 5 | ple 6 |
| Overall mean | 500 | 50 | 1000 | 100 | 200 | 100 |
| dialysis fluid | | | | | | |
| flow at the | | | | | | |
| dialyser | | | | | | |
| (ml/min) | | | | | | |
| Overall mean | 500 | 50 | 50 | 100 | 200 | 100 |
| dialysis fluid | | | | | | |
| flow from the | | | | | | |
| dialysis fluid | | | | | | |
| source | | | | | | |
| (ml/min) | | | | | | |
| Overall mean | 500 | 50 | 50 | 100 | 200 | 100 |
| dialysis fluid | | | | | | |
| flow to the | | | | | | |
| drain (ml/min) | | | | | | |
| Duty Cycle (%) | 50% | 5% | 5% | 10% | 20% | 10% |
| Dwell Period | 0 | 90% | 90% | 80% | 60% | 80% |
| Utilisation of | 0 | 0 | 100% | 100% | 100% | 50% |
| Dwell Period | | | | | | |

Whilst arrangements have been described as including the first flow balance pump 50 and the second flow balance pump 60, other types of pumps may be additionally or alternatively used to achieve the same or similar effect.

Further, or instead, the valve arrangement used will depend upon the type of pump used in the system.

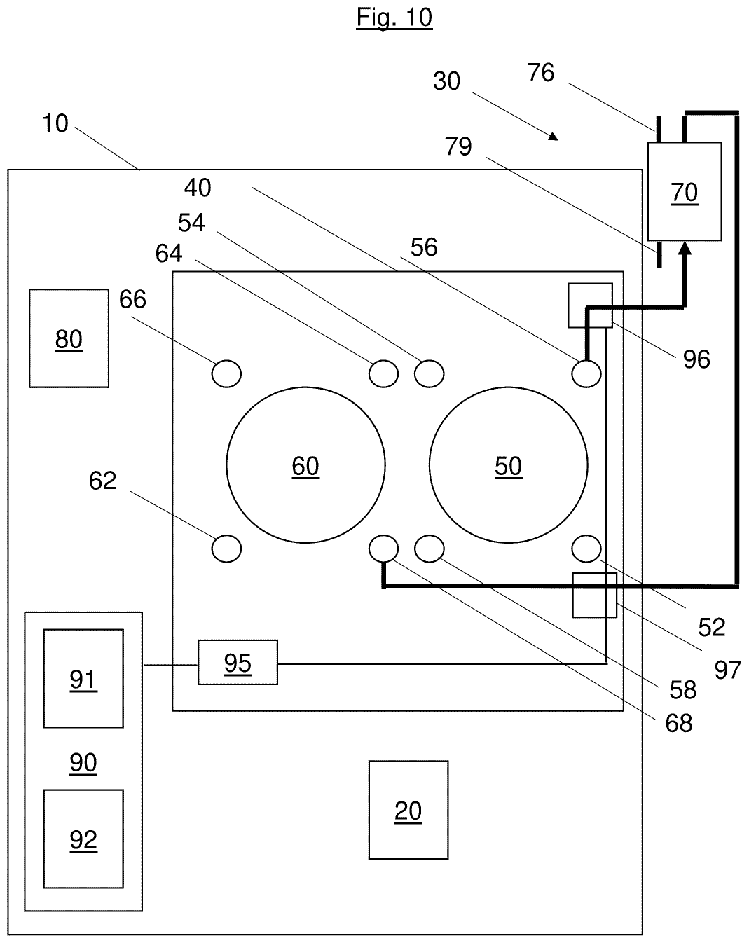

FIG. 11 is a system 510 including a double-sided piston pump 5555. Similar reference numbers are used to depict similar features with respect to the embodiment of FIG. 1, prefixed by the number “5” to indicate that those features relate to a single pump arrangement.

The double-sided piston pump 5555 is operable to deliver a first volume of dialysis fluid from the dialysis source 520 to the dialyser 570 in an inlet cycle. The double-sided piston pump 5555 is operable to remove a second volume of dialysis fluid from the dialyser 570 to a drain 580 in an outlet cycle. A controller 590 is in electrical communication with the double-sided piston pump 555 and operable to operate the double-sided piston pump 555 in the inlet cycle, and to operate the double-sided piston pump 555 in the outlet cycle, wherein for each inlet cycle there is a corresponding outlet cycle. The controller 590 is further operable to operate the piston pump 555 and to circulate dialysis fluid from the dialyser 570 to the piston pump 555 and back to the dialyser 570 in a circulation cycle occurring between completion of the inlet cycle and the outlet cycle, wherein circulation occurs in a single flow direction throughout a given circulation cycle.

The double-sided piston pump 555 has a first side 502 and a second side 504.

Each of the first side 502 and the second side 504 has a dialysis fluid source inlet valve 506, a drain valve 508, a dialyser inlet valve 556, 566 (respectively), and a dialyser outlet valve 558, 568 (respectively).

Fresh dialysis fluid is pumped from first side 502 to the dialyser 570 via dialyser inlet valve 556, by actuation of the double-sided piston pump 555. Spent dialysis fluid is drawn from the dialyser 570 into the second side 504 via dialyser outlet valve 568, by actuation of the double-sided piston pump 555.

Fresh dialysis fluid is drawn from dialysis fluid source 520 into the first side 502 via the dialysis fluid source inlet valve 506, by actuation of the double-sided piston pump 5555. The spent dialysis fluid may be pumped from the second side 504 to the drain 580 via the drain valve 508, by the same actuation of the double-sided piston pump 555.

Thus, in a first step (step 1), first side 502 empties fresh dialysis fluid to the dialyser 570 whilst second side 504 fills with spent dialysis fluid from the dialyser 570. In a second step (step 2), first side 502 fills with fresh dialysis fluid from the dialysis fluid source 520 whilst second side 504 empties spent dialysis fluid to the drain 580.

At the end of step 1, the first side 502 is empty, and the second side 504 is full of spent dialysis fluid.

In circulation step 1, the spent dialysis fluid is pumped from the second side 504 to the dialyser 570 via dialyser inlet valve 566 by actuation of the double-sided piston pump 555, whilst spent dialysis fluid is drawn from the dialyser 570 into the first side 502 via the dialyser outlet valve 558, by the same actuation of the double-sided piston pump 555.

Thus, the spent dialysis fluid is pumped back into the dialyser 570, but in the same flow direction as fresh dialysis fluid. This maintains the counter-current flow of dialysis fluid and blood.

At the end of circulation step 1, first side 502 is full of spent dialysis fluid, and second side 504 is empty.

In circulation step 2, the spent dialysis fluid is pumped from first side 502 to the dialyser 570 via the dialyser inlet valve 556, by actuation of the double-sided piston pump 555. Spent dialysis fluid is drawn from the dialyser 570 into the second side 504 via the dialyser outlet valve 568, by the same actuation of the double-sided piston pump 555.

Thus, again, the spent dialysis fluid is pumped back into the dialyser 570, but in the same flow direction as fresh dialysis fluid. This maintains the counter-current flow of dialysis fluid and blood (or blood analogue fluid).

In the circulation steps 1 and 2, the first side 502, the second side 504 and the dialyser 570 define a closed circuit, fluidically isolated from the dialysis fluid source 520 and the drain 580.

At the end of circulation step 2, first side 502 is empty, and second side 504 is full of spent dialysis fluid, in an analogous condition to the beginning of circulation step 1. Because the pump side conditions are the same, circulation steps 1 and 2 may be repeated.

Since both sides 502, 504 of the piston pump 555 may be provided with a source valve and a drain valve, pump swapping may also be used.

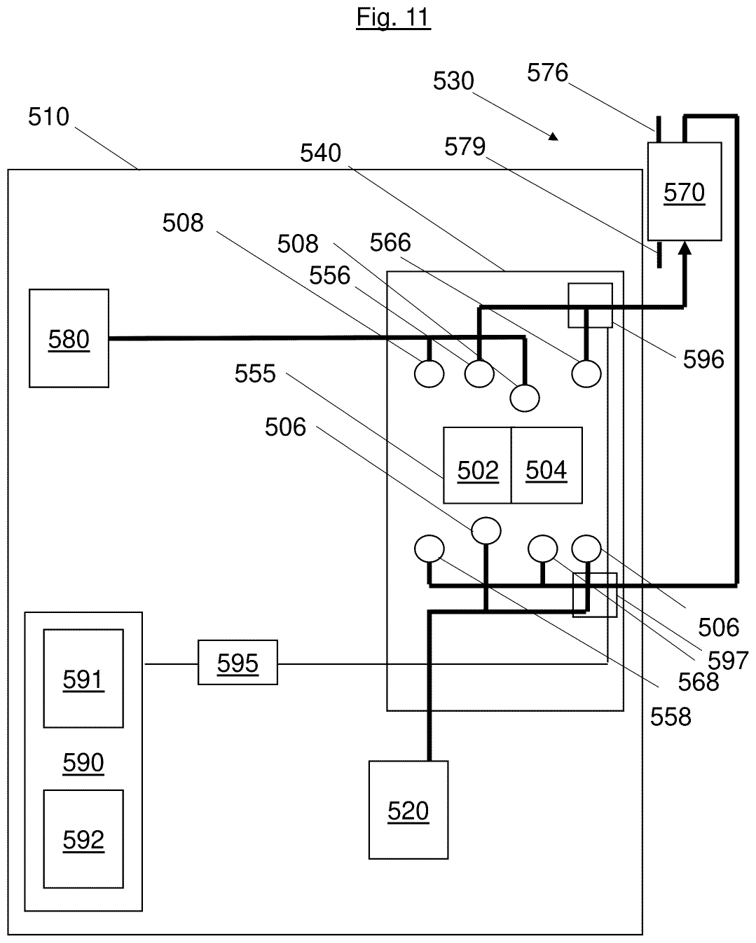

FIG. 12 shows a peristaltic pump 655. Similar reference numbers are used to depict similar features with respect to the embodiment of FIG. 1, prefixed by the number “6” to indicate that those features relate to a peristaltic pump arrangement.

Given the nature of the peristaltic pump 655, flow may be achieved using only a dialysis fluid source inlet valve 652 and a drain valve 654, although it shall be appreciated that additional valves may also be used.

A gear pump may be used in the same way as the peristaltic pump 655. A vane pump may be used in the same way as the peristaltic pump 655. An impeller pump may be used in the same way as the peristaltic pump 655. A combination of pump types may be used.

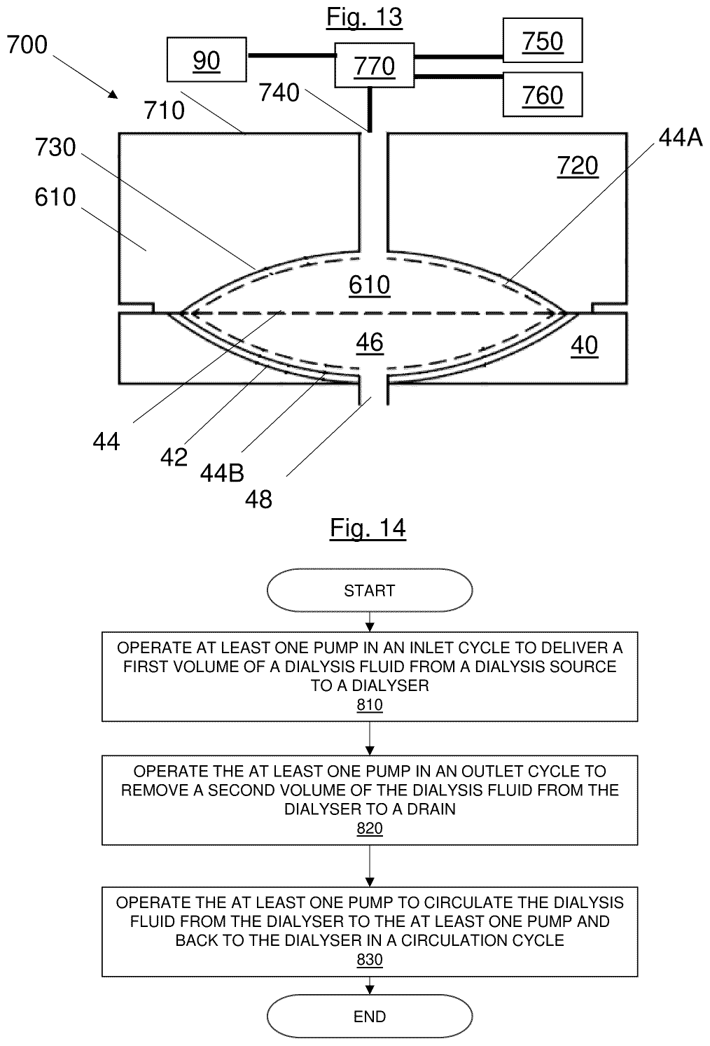

FIG. 14 is a flowchart of a method 800 of pumping dialysis fluid in accordance with an example implementation. At block 810 the pump inlet cycle is performed. In an example the at least one pump is operated to draw a first volume of dialysis fluid into the at least one pump from the fluid source 20, and the first volume of dialysis fluid is delivered to the dialyser 70. At block 820 the pump outlet cycle is performed. In an example the at least one pump is operated to remove a second volume of dialysis fluid from the dialyser 70 and pumped to drain 80. At block 830 the pump circulation cycle is operated. In an example dialysis fluid is circulated between the dialyser 70 to the at least one pump and back to the dialyser 70 in a circulation cycle isolated from the dialysis fluid source 20 and the drain 80.

Whilst the embodiments above have been described as arrangements utilising membrane pumps and membrane valves, the same effect may be achieved with alternate pump and valve arrangements, such as peristaltic (linear or rotary) or positive displacement pumps.

What is claimed is:

1. A system for pumping dialysis fluid, the system comprising:
a dialyser having a semi-permeable membrane;
at least one pump in fluid communication with the dialyser, the at least one pump operable to deliver a first volume of dialysis fluid from a fluid source to the dialyser in an inlet cycle, the at least one pump operable to remove a second volume of dialysis fluid from the dialyser and deliver the second volume of dialysis fluid to a drain in an outlet cycle; and
a controller in electrical communication with the at least one pump, the controller configured to:
operate the at least one pump in the inlet cycle and in the outlet cycle,
operate the at least one pump to circulate dialysis fluid from the dialyser to the at least one pump and back to the dialyser in a circulation cycle isolated from the fluid source and the drain.

2. The system of claim 1, wherein the controller is configured to operate the at least one pump with each instance of the inlet cycle corresponding to a respective instance of an outlet cycle.

3. The system of claim 1, wherein the controller is configured to operate the at least one pump in the circulation cycle between completion of the inlet cycle and the outlet cycle.

4. The system of claim 1, wherein operation of the at least one pump in the circulation cycle circulates the dialysis fluid through the dialyser in a single flow direction throughout the circulation cycle.

5. The system of claim 1, further comprising a blood pump, wherein the semi-permeable membrane divides the dialyser into a blood side and a dialysis fluid side, wherein the blood pump is in fluid communication with the blood side of the dialyser as part of a blood circuit, and the controller is configured to actuate the blood pump such that fluid flows through the blood side in a direction opposite to a flow direction in which dialysis fluid circulates through the dialysis fluid side of the dialyser throughout the circulation cycle of the at least one pump.

6. The system of claim 1, wherein the controller is configured to operate the at least one pump in one or more circulation cycles between the inlet cycle and the outlet cycle.

7. The system of claim 1, wherein the controller is configured to operate the at least one pump in the circulation cycle a number of times, n, in between completion of the inlet cycle and the outlet cycle, wherein n is greater than or equal to 2.

8. The system of claim 1, wherein the controller is configured to operate the at least one pump in the circulation cycle a predetermined number of times in between completion of the inlet cycle and the outlet cycle, wherein the predetermined number may vary according to at least one of treatment type, treatment length, and/or dialysis fluid composition.

9. The system of claim 1, wherein the at least one pump is defined in part by a flexible membrane, the flexible membrane being independently operable between an open position and a closed position.

10. The system of claim 1, wherein the at least one pump is operable to move a fluid volume at a volumetric flow rate of 50 to 1000 ml/minute through the at least one pump.

11. A method of pumping dialysis fluid, the method comprising the steps of:
operating at least one pump in an inlet cycle to deliver a first volume of a dialysis fluid from a dialysis fluid source to a dialyser having a semi-permeable membrane;
operating the at least one pump in an outlet cycle to remove a second volume of the dialysis fluid from the dialyser to a drain; and
operating the at least one pump to circulate the dialysis fluid from the dialyser to the at least one pump and back to the dialyser in a circulation cycle isolated from the dialysis fluid source and the drain, wherein the circulation cycle occurs between completion of the inlet cycle and the outlet cycle, and wherein the circulation cycle occurs in a single flow direction along the semi-permeable membrane to clean the semi-permeable membrane.

12. The method of claim 11, further comprising
operating at least one pump in an inlet cycle to deliver a first volume of a dialysis fluid from a dialysis fluid source to a dialyser having a semi-permeable membrane comprises operating a first pump,
operating the at least one pump in an outlet cycle to remove a second volume of the dialysis fluid from the dialyser to a drain comprises operating a second pump, and
operating the at least one pump to circulate the dialysis fluid from the dialyser to the at least one pump and back to the dialyser in a circulation cycle isolated from the dialysis fluid source and the drain comprises operating the first pump and the second pump to circulate the dialysis fluid from the dialyser to a first one of the first pump and the second pump and back to the dialyser, and to circulate dialysis fluid to the dialyser from another one of the first pump and the second pump and back to the other one of the first pump and the second pump in a circulation cycle isolated from the dialysis fluid source and the drain.

13. The method of claim 11, further comprising
operating a blood analogue pump, wherein the semi-permeable membrane divides the dialyser into a blood analogue side and a dialysis fluid side, wherein the blood analogue pump is in fluid communication with a blood side of the dialyser as part of a blood analogue circuit, wherein operating the blood analogue pump causes a blood analogue fluid to flow through the blood analogue side in a flow direction opposite to the single flow direction in which the dialysis fluid circulates throughout the circulation cycle.

14. The method of claim 11, further comprising the step of reversing the single flow direction in which dialysis fluid circulates through the dialyser in a further circulation cycle.

15. A controller for controlling pumping of dialysis fluid, the controller comprising:
a processing unit; and
one or more non-transitory computer-readable storage media in electrical communication with the processing unit, the one or more non-transitory computer-readable storage media having stored thereon computer-readable instructions that, when executed by the processing unit, cause the processing unit to carry out steps including
operating at least one pump in an inlet cycle to deliver a first volume of a dialysis fluid from a dialysis fluid source to a dialyser having a semi-permeable membrane,
operating the at least one pump in an outlet cycle to remove a second volume of the dialysis fluid from the dialyser to a drain, and
operating the at least one pump to circulate the dialysis fluid from the dialyser to the at least one pump and back to the dialyser in a circulation cycle isolated from the dialysis fluid source and the drain, wherein the circulation cycle occurs between completion of the inlet cycle and the outlet cycle, and wherein the circulation cycle occurs in a single flow direction along the semi-permeable membrane to clean the semi-permeable membrane.
